(19) United States
(12) Patent Application Publication
(10) Pub. No.: US 2010/0168504 A1
(43) Pub. Date: Jul. 1, 2010

(54) METHOD AND APPARATUS FOR THE AID OF MEDITATION AND HYPNOSIS

(76) Inventors: Jaime Alfredo Castano, SR., Spring, TX (US); Charles William Mathias, JR., Spring, TX (US)

Correspondence Address: Jaime Castano, 2083 Five Oaks Drive, Missouri City, TX, 77459, US

(21) Appl. No.: 12/647,520

(22) Filed: Dec. 27, 2009

Related U.S. Application Data
(60) Provisional application No. 61/140,970, filed on Dec. 28, 2008.

(51) Int. Cl. A61M 21/00

(52) U.S. Cl. 600/27

(57) ABSTRACT

An apparatus that aids a user to reach a meditative or hypnotic state. The apparatus includes a reflector. Once a user is in a meditative or hypnotic state, the user can speak directly to their subconscious while looking at him or herself in the reflector, whether repeating a healing mantra or stating positive affirmations. A user can also look at him or herself in the reflector while listening to a hypnotist or to a recording of a guided meditation thereby significantly amplifying the effectiveness of the entire therapy session.

### CROSS-REFERENCE TO RELATED APPLICATION

This application claims priority, pursuant to 35 U.S.C. §119(e), to the filing date of U.S. Provisional Patent Application Ser. No. 61/140970, entitled “Methods and Apparatus for the Aid of Meditation and Hypnosis,” filed on Dec. 28, 2008, which is hereby incorporated by reference in its entirety.

### BACKGROUND

Millions of people the world over meditate each day using a variety of methods and for a variety of reasons. But whatever the method or the specific aim of the meditation, the one common denominator is that people are seeking something that is not normally accessible to them in the ordinary waking state. From the earliest of times, almost all religions practiced meditation as a means of achieving an altered state of consciousness, whether to experience ecstatic union with God, to cultivate a loving heart, or simply to find peace. In recent times, secular practitioners have turned to meditation to achieve such benefits as greater mental focus, creative insight or inspiration, relief from stress, and the healing of a variety of other health concerns.

In his 1975 book The Relaxation Response, Herbert Benson, M.D., made the claim that meditation, whether religious or secular, is essentially about eliciting the “relaxation response.” The opposite of the “fight or flight response,” the relaxation response that occurs during meditation involves certain basic physiological changes, including a decrease in metabolism (oxygen consumption), decrease in respiratory rate, lower heart rate, lower blood pressure, and an increase in alpha brain waves.

Hypnosis, whether induced by a hypnotist of self-induced, is an altered state of consciousness characterized by a heightened responsiveness to suggestion. Hypnosis has a long tradition of effective practice in the fields of both psychology and medicine. In the medical field it has been used to perform painless surgery and painless dental procedures—in fact, both the British and American Medical Associations have made statements supporting the usefulness of hypnosis.

In the field of psychology, hypnosis has proven an effective therapy for phobias, for regression to uncover past traumas, for panic attacks and generalized anxiety, for stress reduction, for boosting self-confidence, for quitting smoking, losing weight, and a host of other problems.

The effectiveness of hypnosis depends on the hypnotist's ability to induce in the patient a state of consciousness in which the subconscious mind is open to the suggestions the hypnotist is making. Although the conscious mind only accounts for about five percent of the mind, it is normally in control during the waking state and guards access to the subconscious mind rather jealously. The subconscious mind, about 95 percent of the mind, involves our emotions, imagination, long-term memories, and autonomic nervous system.

Self-hypnosis, whether using a relaxation technique, following one's breathing, repeating a mantra, or staring at a single point or a mandala, is very like meditation. The primary difference is that self-hypnosis, like hypnosis, employs positive suggestions or affirmations to direct the subconscious mind toward a desired goal. When relaxed to a point in which alpha brain waves are present, as they are in meditation, a person will be in a “hypnotic state” insofar as his or her conscious mind will be relaxed enough to let down its guard so the subconscious mind may be engaged directly. Since it is awkward to give yourself suggestions or affirmations while you are meditating or in a hypnotic state, it is best to use a pre-recorded guided meditation or script that you have written yourself and recorded for playback.

Prior art hypnosis or meditative aids include visual and aural aids. Visual aids include posters with mandalas for the eyes to focus on until a meditative state is reached. A mandala is a geometric or pictorial design that is often enclosed in a circle and used to facilitate meditation. Video tapes may also use the mandala or other images together with relaxing, hypnotic music accompanied by subliminal suggestions or spoken affirmations or both.

Aural aids include recordings that use binaural beats. For meditation purposes, binaural beats generally stimulate alpha brain waves (alpha brain waves are associated with relaxation). Due to a process called entrainment, the predominant brain wave frequency, generally the alert, awake beta frequency, will move toward the frequency of the stimulus. Headphones are required.

Other aural aids are simple recorded guided meditations, recorded self-hypnosis scripts, or relaxing music with or without subliminal messages. The spoken words together with soothing background music help induce a relaxed state of mind. Self-hypnosis scripts may include positive affirmations and/or other language aimed at the subconscious mind for the purpose of healing or self-improvement.

None of the prior art meditative or hypnotic aids described above, taken either singly or in combination, is seen to describe the instant invention as claimed.

### SUMMARY

In accordance with one example, the invention comprises an apparatus to aid a person reach a meditative state, comprising a reflector and a structure, with the reflector in functional relationship with the structure. In accordance with another example, the invention comprises an apparatus to aid a person reach a meditative state, comprising a mirror and a hollow structure that houses at least the person's head so that the person is in visual contact with the mirror.

Other or alternative features will become apparent from the following description, from the drawings, and from the claims.

### BRIEF DESCRIPTION OF THE DRAWINGS

FIG. 1 is a schematic of one embodiment of the invention.

FIG. 2 is a schematic of an alternative embodiment of the invention.

FIG. 3 is a schematic of an alternative embodiment of the invention.

FIG. 4 is a schematic of an alternative embodiment of the invention.

FIG. 5 is a schematic of an alternative embodiment of the invention.

FIG. 6 is a schematic of an alternative embodiment of the invention.

FIG. 7 is a schematic of an alternative embodiment of the invention.

### DETAILED DESCRIPTION

In the following description, numerous details are set forth to provide an understanding of various embodiments. However, it will be understood by those skilled in the art that the other embodiments may be practiced without these details and that numerous variations or modifications from the described embodiments are possible.

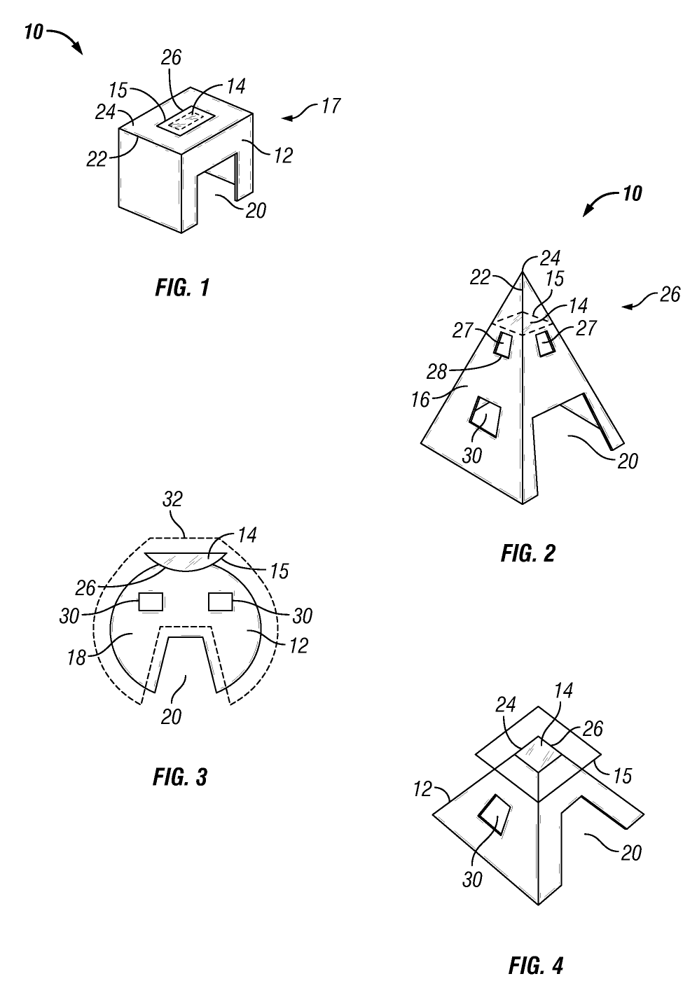

The apparatus of this invention aids a person reach a meditative or hypnotic state. For purposes of brevity, the terms “meditative” or “meditation” as used in this patent application will be understood to include the states of “hypnosis” and “self-hypnosis.” FIG. 1 generally shows the apparatus of this invention at 10. The apparatus 10 comprises a structure 12 and a reflector 14, with the reflector 14 in functional relationship with the structure 12. In one embodiment, the structure 12 supports the reflector 14.

The structure 12 can be constructed from any material strong enough to support the weight of the apparatus 10. For instance, the structure 12 may be constructed from cardboard, wood, metal, or plastic.

The reflector 14 may comprise any reflective surface 15 that can reflect an image, such as a person's face situated directly in front of the reflector 14. For instance, the reflector 14 may comprise a mirror, metal surface, or other polished surface.

The structure 12, although shown as a box 17 in FIG. 1, can have any shape. For example, the structure 12 can also be a pyramid 16 (FIG. 2), a sphere 18 (FIG. 3), or a cylinder 19 (FIG. 6). The structure 12 partially or completely covers a person while in use. For instance, as shown in FIG. 2, the structure 12 can include an opening 20 through which a person may insert his or her head. The structure 12 may also be hollow so that a person can insert all of his or her head therein. Depending on the size of the structure 12, the person may also be able to insert some or all of his or her shoulders. In another embodiment as shown in FIG. 6, the structure 12 completely covers a person.

In one embodiment as in FIGS. 1-6, the reflector 14 is attached to the structure 12. In another embodiment as in FIG. 7, the reflector 14 is in functional relationship with but is not attached to or supported by the structure 12. For instance, in the embodiment of FIG. 7, the reflector 14 may be attached to a support 15 (which may itself include reflective surfaces) that is on the ground proximate to the person thereby being in functional relationship with the structure 12.

For the embodiment in which the reflector 14 is supported by the structure 12, the reflector 14 can be attached to the top 24 of the structure 12, such as on its underside 22, so that, when a person's head is within the head opening 20, the person's eyes are in visual contact or optical communication with the reflector 14. The reflective surface 15 of the reflector 14 is therefore, in one embodiment, facing downwards towards the person's head. Attachment of the reflector 14 to the top 24 of the structure 12 can be accomplished by any suitable methods such as glue, tape, fasteners, or a tongue and groove mechanism. In the embodiment of FIG. 6 where a person's entire body is inside of the structure 12, the reflector 14 may be attached to the side 21 of the structure 12 so that the person is in visual contact with the reflector 14 while sitting within the structure 12. Attachment of the reflector 14 to the side 21 of the structure 12 can be accomplished by any suitable methods such as glue, tape, fasteners, or a tongue and groove mechanism.

In another embodiment such as shown in FIG. 1, the top 24 of the box structure 17 can have a reflector opening 26 and the reflector 14 can be temporarily supported on the structure 12 such as by placement of the reflector 14 on the top 24 with the reflective surface 15 facing the downward position. Similarly, for the embodiment of FIG. 3, the top 24 of the sphere structure 18 can have a reflector opening 26 and the reflector 14 can be temporarily supported on the structure 12 such as by placement of the reflector 14 on the top 24 with the reflective surface 15 facing the downward position. For the embodiment shown in FIG. 4, the pyramid structure 16 can have a reflector opening 26 and the reflector 14 can be temporarily supported on the structure 12 such as by placement of the reflector 14 on the top 24 with the reflective surface 15 facing the downward position. Alternatively for the pyramid structure 16 and as shown in FIG. 2, the reflector opening 26 can comprise multiple reflector openings 27, such as but not limited to at least one opening 27 on each side of the pyramid 16. In this alternative embodiment, the reflector 14 can be temporarily supported on the bottom edge 28 of each reflector opening 27 with the reflective surface 15 facing the downward position. In any of these embodiments, the reflector 14 may be secured in its position at the top 24 of the structure 12 with tape or a fastener to ensure it does not fall while the apparatus 10 is in use.

The reflector 14, in one embodiment, is sized and is located a distance “D” from the person's eyes so that the person can see his or her eyes on the reflective surface 15 of the reflector 14. Alternatively, the reflector 14 is sized and located from the person's eyes so that the person can see his or her entire face or body on the reflective surface 15 of the reflector 14.

In one embodiment, the structure 12 may include a plurality of air openings 30 that allow air to flow therethrough thereby making the environment within the structure 12 more comfortable for the person. The multiple reflector openings 27 of the embodiment shown in FIG. 2 also act as air openings 30 allowing air to flow therethrough.

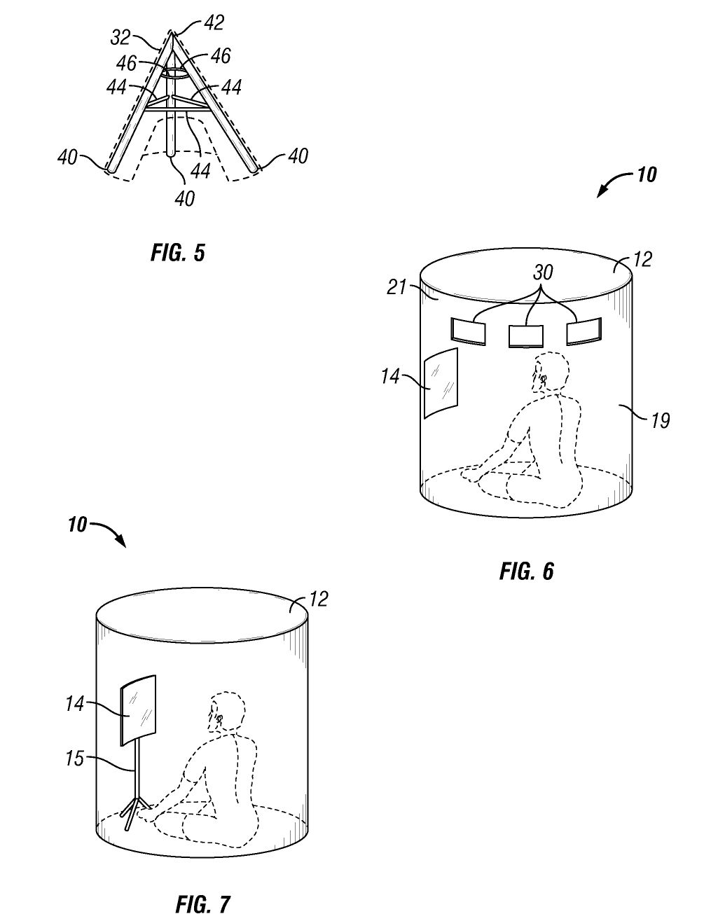

In an alternative embodiment as shown in FIG. 5, the structure 12 comprises three or more legs 40 pivotally connected together at one of their ends 42 to form a pyramid shape when the legs 40 are pivoted away from each other. Braces 44 between adjacent legs 40 prevent the legs 40 from being pivoted beyond an acceptable angle. Once the legs 40 are pivoted, a cover 32 can be placed over the structure 12. In this embodiment, the reflector 14 may be sewn into the cover 32 or it may be laid on top of support braces 46 near the pivoted ends 42.

Any of the pyramid shapes (FIGS. 2, 4, and 5) may be three or four sided, depending on the desire of the user and the materials available to the user for construction.

In use, and for the embodiments of FIGS. 1-5, a person may lie horizontally on the ground and may insert his or her head into the head opening 20 of the structure 12. The person lies on his or her back so that the eyes of the person face upwards. The person's eyes are then in visual contact or optical communication with the reflective surface 15 of the reflector 14. Alternatively, in the embodiment of FIG. 6, a person enters the structure 12 (such as through an opening not shown) or picks up the structure 12 and places it around him or herself. The person then sits inside of the structure 12 and establishes visual contact with the reflector 14 on the side 21 of the structure 12.

In any of the illustrated embodiments, the apparatus 10 may also include a cover 32 that is placed on top of and to cover the structure 12 and reflector 14. The cover 32, in one embodiment, covers all of the openings 27, 30 of the structure 12 and suppresses some light from entering the interior of the structure 12 while allowing sufficient air to flow therethrough and through the air openings 30. The suppression of light to the interior of the structure 12 makes the environment within the structure 12 more conducive to reaching a meditative state. In the embodiment of FIG. 5, cover 32 may include the opening 30 through which a person inserts his or her head.

The apparatus 10 can aid in any style of meditation, including mindfulness meditation, insight meditation, transcendental meditation, meditation for relaxation, or healing meditation. The apparatus 10 is particularly useful with healing a broad range of symptoms, addictions, and neuroses in combination with hypnosis or self-hypnosis. The aim of the apparatus 10 is for the user to generate alpha brain waves, since the presence of alpha brain waves indicates that the user's subconscious is susceptible to suggestions (as in hypnosis).

In operation, a person uses the apparatus 10 to reach a meditative or hypnotic state. The user looks at him or her self in the reflector 14 and repeats a healing mantra or positive affirmations. The user thereby reaches a connection (and speaks directly) with his or her subconscious. The user can also simply look at him or herself in the reflector 14 while listening to a hypnotist or to a recording of a guided meditation. Looking in the mirror while repeating positive affirmations, for instance, significantly amplifies the effectiveness of the entire therapy session. Hypnotism can accomplish extraordinary things for patients, such as help with weight loss, quitting smoking, anxiety attacks, stress, self-confidence, emotional healing, or spiritual growth. The apparatus 10 enables any user to achieve these same results through self-meditation or hypnosis at any location. The apparatus 10 also limits the user's vision to only the user's image in the reflector; thus separating the user from the outside world that is the major cause of distraction in the meditation process.

Although certain example methods and apparatus have been described herein, the scope of coverage of this patent is not limited thereto. On the contrary, this patent covers all methods, apparatus and articles of manufacture fairly falling within the scope of the appended claims either literally or under the doctrine of equivalents.

What is claimed is:

1. An apparatus to aid a person reach a meditative state, comprising:
a structure and a reflector in functional relationship with the structure.

2. The apparatus of claim 1, wherein the structure supports the reflector.

3. The apparatus of claim 1, wherein the reflector is a mirror.

4. The apparatus of claim 1, wherein the structure at least partially covers the person.

5. The apparatus of claim 4, wherein:
the person has eyes; and
the structure covers the eyes of the person.

6. The apparatus of claim 5, wherein the reflector is in optical communication with the eyes of the person.

7. The apparatus of claim 5, wherein the structure is a pyramid.

8. The apparatus of claim 5, wherein the structure is a sphere.

9. The apparatus of claim 5, wherein the structure is a cylinder.

10. The apparatus of claim 5, wherein the structure is a box.

11. The apparatus of claim 5, wherein:
the structure is hollow and has a top end, and
the reflector is positioned at the top end.

12. The apparatus of claim 11, wherein:
the structure is hollow and has a side and
the reflector is positioned on the side.

13. The apparatus of claim 1 wherein the structure is constructed from cardboard.

14. The apparatus of claim 5, wherein the structure has holes through which air can flow.

15. The apparatus of claim 5, wherein a cover is placed on top of the structure.

16. An apparatus to aid a person reach a meditative state, comprising:
a mirror;
a hollow structure that houses at least the person's head so that the person is in visual contact with the mirror.

17. The apparatus of claim 16, wherein the mirror is supported by the structure.

18. The apparatus of claim 16, wherein the person lies in a horizontal position

19. The apparatus of claim 16, wherein the structure houses the person's entire body

20. The apparatus of claim 16, wherein the structure is constructed from cardboard
